(19) United States
(12) Patent Application Publication
(10) Pub. No.: US 2022/0152195 A1
(43) Pub. Date: May 19, 2022

(54) METHODS OF PREVENTING AND TREATING VIRAL INFECTIONS

(71) Applicant: Leonhardt Ventures LLC, Mission Viejo, CA (US)

(72) Inventors: Howard J. Leonhardt, Mission Viejo, CA (US); Jorge Genovese, Buenos Aires (AR)

(21) Appl. No.: 17/526,698

(22) Filed: Nov. 15, 2021

Related U.S. Application Data
(60) Provisional application No. 63/113,612, filed on Nov. 13, 2020.

(51) Int. Cl. A61K 39/215; A61N 1/36; A61P 31/14; A61K 35/28; A61H 1/00

(52) U.S. Cl. CPC A61K 39/215; A61N 1/36014; A61N 1/36034; A61K 2039/53; A61K 35/28; A61H 1/00; A61P 31/14

(57) ABSTRACT

Described are methods of treating a mammalian subject who is intending to undergo exposure to an inoculant comprising a virus, polynucleotide(s) encoding at least a portion of the virus, and/or epitope(s) of the virus, the method including administering at least one bioelectric signal to the subject before exposure to the inoculant in such a manner as to increase the subject's T cell count and/or T helper cell count. Also described are methods of treating a mammalian subject undergoing a viral infection, the method comprising: administering bioelectric signals to the subject so as to upregulate expression of SDF-1 in the subject, upregulate expression of PDGF in the subject, upregulate stem cell proliferation in the subject, and upregulate expression of klotho in the subject; reducing inflammation in the subject, and administering bioelectric signals to the subject so as to stimulate regeneration of the subject's lungs and blood vessels.

### CROSS-REFERENCE TO RELATED APPLICATION

This application claims the benefit under 35 U.S.C. § 119(e) of U.S. Provisional Patent Application Ser. No. 63/113,612, filed Nov. 13, 2020, and entitled “METHODS OF PREVENTING AND TREATING CORONAVIRUS USING T CELL IMMUNITY,” the disclosure of which is hereby incorporated herein in its entirety by this reference.

### TECHNICAL FIELD

This application relates generally to the treatment and prevention of infections, such as viral infections, and more specifically relates to the treatment and prevention of “coronavirus” or SARS-CoV-2 in a mammalian subject.

### BACKGROUND

As the COVID-19 epidemic sweeps the world, scientists are busy developing vaccines against SARS-CoV-2. Unfortunately however, concerns are being raised that the antibodies produced by a vaccine may not last long enough to serve as a good, long term preventative to COVID-19. See, e.g., Mary Van Beusekom, “Study: COVID-19 antibodies decay quickly after mild illness,” CIDRAP News (Jul. 22, 2020), where it was reported that antibodies against SARS-CoV-2 (the virus that causes COVID-19) were dramatically reduced over the first 3 months of infection in 34 people who had recovered from mild illness. See, also, Alexander McNamara, “Coronavirus: antibody immunity could last ‘just months,’” BBC Science Focus (Jul. 13, 2020) reporting “a significant drop in antibody potency after three months.” These early results do not bode well for “immunity passports,” herd immunity, and vaccines.

Partially due to these early observations, there has also been effort put into researching T cell immunity; not just antibody immunity. See, e.g., F. Collins, “Immune T Cells May Offer Lasting Protection against COVID-19,” NIH Director's Blog, directorsblog.nih.gov/2020/07/28/immune-t-cells-may-offer-lasting-protection-against-covid-19/. See also, R. Rettner, “Common colds train the immune system to recognize COVID-19,” www.livescience.com/common-cold-coronaviruses-t-cells-covid-19-immunity.html (August 2020). See, also, Sekine et al., “Robust T cell immunity in convalescent individuals with asymptomatic or mild COVID-19,” bioRxiv (Jun. 29, 2020), which described that SARS-CoV-2 induces robust memory T cell responses in antibody-seronegative and antibody-seropositive individuals with asymptomatic or mild COVID-19.

Unfortunately however, after continued assault by SARS-CoV-2 on that aspect of the subject's immune system, T cells ultimately may be reduced and experience “exhaustion.” See, e.g., Diao et al., “Exhaustion of T Cells in Patients with Coronavirus Disease 2019 (COVID-19),” Front. Immunol., vol. 11, p. 827 (May 1, 2020); 10.3389/fimmu.2020.00827.

Interleukin 2 (“IL-2”) was one of the first cytokines to be discovered, and research is being conducted in determining the complex role it plays in the body. See, e.g., Bachmann et al., “Interleukin 2: from immunostimulation to immunoregulation and back again,” EMBO Rep. 2007 December; 8(12): 1142-1148; doi: 10.1038/sj.embor.7401099. See, also Khan et al., “The Timing of Stimulation and IL-2 Signaling Regulate Secondary CD8 T Cell Responses,” PLoS Pathog. 2015 October; 11(10): e1005199. IL-2 has been identified as a T cell growth factor.

As reported by Zhang et al., “Potential contribution of increased soluble IL-2R to lymphopenia in COVID-19 patients,” Cell Mot Immunol. 17, 878-880 (2020), “the mechanism of cytokine-induced lymphopenia in COVID-19 is very unclear. IL-2 is critical for the proliferation, differentiation, and function of T cells, including Tregs, CD4+, and CD8+ effector cells.” Zhang et al., reported the negative relationship between the concentration of soluble IL-2 receptor (sIL-2R) and T-cell number in blood from COVID-19 patients, and that their “data suggested the importance of IL-2 signaling in lymphopenia of COVID-19 patients.”

It has also been found that priming killer T cells in the presence of Interleukin 12 (“IL-12”) enhances their function. “Scientists discover way to amp up power of killer T cells to fight melanoma,” Science Daily (May 11, 2011).

### BRIEF SUMMARY

Described herein is a treatment comprising strengthening the patient's immune system and reducing inflammation and oxidative stress via bioelectric stimulation (application of bioelectric signals) so as to control (e.g., upregulate or downregulate) expression of, for example, IL-2, klotho, PDGF, and other proteins in the patient. Such a treatment may be combined with the application of harmonic vibrational energy delivered into the patient's lungs to prevent blood clot aggregation. Stimulating increased IL-2 and Klotho protein expression in the patient increases T cell and T helper cells production and reduces inflammation in order to kill the invading virus before lung damage, blood vessel damage or clots occur.

Specifically described herein is a method of treating a mammalian subject who is about to undergo exposure to an inoculant comprising a virus, polynucleotide(s) encoding at least a portion of the virus, and/or epitope(s) of the virus, the method comprising: administering at least one bioelectric signal to the subject before, during, or after exposure to the inoculant in such a manner as to increase the subject's T cell count and/or T helper cell count.

Such a method can further include inoculating the subject with the inoculant after administration of the at least one bioelectric signal and after the subject has experienced an increased T cell count and/or T helper cell count so as to create specific memory T cells against the virus. In such a method, the virus is preferably SARS-CoV-.

In such a method, the bioelectric signal typically originates from a bioelectric stimulator programmed to produce at least one bioelectric signal. In certain embodiments, the bioelectric signal may be self-administered by the subject.

In such a method, the bioelectric signal may be applied in the location of the subject's thyroid.

In such a method, the bioelectric signal preferably upregulates the expression of interleukin-2 (“IL-2”) and/or interleukin-2 (“IL-2”) by the subject.

In such a method, at least one bioelectric signal may upregulate klotho expression.

In such a method, at least one bioelectric signal may upregulate expression of Stromal Cell-Derived Factor 1 (“SDF-1”).

In such a method, at least one bioelectric signal may upregulate Sonic Hedgehog Expression. See, e.g., Hanna et al., “Evaluation of the Role of Hedgehog Interacting Protein (HHIP) and the Sonic Hedgehog Pathway to Enhance Respiratory Repair and Function in Chronic Obstructive Pulmonary Disease (COPD),” American Journal of Respiratory and Critical Care Medicine 2020; 201:A4062, the contents of which are incorporated herein by this reference. Such bioelectric signals are described in U.S. Patent Application Publication US 2020-0324106-A1 to Leonhardt et al. (Oct. 15, 2020) for “Bioelectric Stimulation for Sonic Hedgehog Expression,” the contents of which are incorporated herein by this reference.

Such a method may further include administering to the subject a material that stimulates an immune response to SARS-CoV-2.

Such a method may further include administering to the subject a material that enhances function of T cells and/or reduces T cell exhaustion.

Such a method may further include administering to the subject material inducing an immune response against spike protein of SARS-CoV-2. Such a material may be delivered, e.g., by adenovirus, such as Ad26.

In such a method, the inoculant may be a polynucleotide comprising mRNA.

If COVID-19 has already taken hold in the patient, the treatment method differs from the foregoing “inoculation method.” In such a case, run-away inflammation must be kept in check, and blood clotting kept under control. Lungs, heart, and blood vessel linings may need to subjected to a regeneration therapy for optimal recovery.

Thus, also described is a method of treating a mammalian subject undergoing a viral infection, the method comprising: administering bioelectric signals to the subject so as to upregulate expression of SDF-1 in the subject, upregulate expression of PDGF in the subject, upregulate stem cell proliferation in the subject, and upregulate expression of klotho in the subject; reducing inflammation in the subject, while also administering bioelectric signals to the subject so as to stimulate regeneration of the subject's lungs and blood vessels.

In such a method, a pharmacological agent may also be administered to the subject to reduce inflammation.

In such a method, at least one bioelectric signal may be administered to the subject for inflammation reduction.

Such a method may further include administering biologic and/or pharmacological therapy to the subject.

In such a method, the virus may be SARS-CoV-2.

Such a method may further include administering a statin or hydroxychloroquine to the subject so as to reduce inflammation or other pharmacologic agents such as estrogen or an ACE inhibitor.

Such a method may further include administering nutrients to the subject, wherein the nutrients are selected from the group consisting of vitamin A, zinc, vitamin C, vitamin E, vitamin K, phytochemicals, carotenoids, polyphenols, vitamin D, vitamin K, dietary fiber, cannabidiol (CBD), and any combination thereof.

Such a method may further include applying harmonic vibrational energy delivered into the patient's lungs to prevent blood clot aggregation.

In certain embodiments, administration of a bioelectric signal or signals to a subject or cell increases the expression of interferon type 1 (IFN-1), interferon β (IFNβ), and/or sphingosine kinase 1 (SPHK1) are upregulated, and/or inhibits the expression of, AKT-1, Angiopoietin 2 (ANGPT-2), B-cell lymphoma 2 (BCL-2), chemokine (C-X-C motif) ligand 9 (CXCL9), chemokine (C-X-C motif) ligand 10 (CXCL10), basic fibroblast growth factor (FGF-β or FGF-2), leptin (LEP), transforming growth factor-beta 2 (TGF-β2), and/or transforming growth factor (TGF-β1).

Voltages used with the described bioelectric signals typically vary from 3 to 20 V and typically produce 2 to 20 mA current as may be measured at the level of the cell being stimulated.

Treatment times typically last from at least 15 minutes, 30 minutes, or a few hours daily to continuous bioelectric therapy during the duration of treatment of the COVID patient. Continuous treatment is preferred.

In certain embodiments, the methods described herein further include the use of a, for example, re-fillable pump (see, e.g., U.S. Patent Application Publication US 20180064935 A1 to Leonhardt et al., the contents of which are incorporated herein by this reference) to continuously infuse into the lung(s) of a severely ill COVID patient a composition comprising, e.g., hypoxia-treated mesenchymal stem cells (“MSCs”), klotho-expressing MSCs (see, e.g., EP 3,262,159 B1 (Jul. 24, 2019) to Gunther et al., the contents of which are incorporated herein by this reference), stromal fraction, lung matrix, exosomes, micro RNA gel, vitamin K, selected alkaloids, nutrient hydrogel, bioelectric treated platelet rich fibrin, amniotic fluid, secretome from amniotic sourcing, Wharton's Jelly, growth factors, and proteins.

In certain embodiments, a method includes applying an approximately 50 Hz signaling and vibrational harmonic energy to stave off blood clot deaths in COVID patients. This may be combined with standard blood thinners and/or at home use of daily baby aspirin (e.g., 80 mg) after returning home. See, e.g., Hoffmann & Gill, “Externally Applied Vibration at 50 Hz Facilitates Dissolution of Blood Clots In-Vitro,” Am. J. Biomed. Sci. 2012, 4(4), 274-284, the contents of which are incorporated herein by this reference.

Also described is a method of selecting bioelectric signaling sequences to treat a subject, the method comprising utilizing Raman spectroscopy RNA light change detection to monitor the subject's cells during treatment with at least one bioelectric signal, and assist in the selection of bioelectric signals to treat the subject. The Raman spectroscopy RNA light change detection may be used to custom design bioelectric signaling sequences for treatment of a subject, such as a subject suffering from COVID-19.

### BRIEF DESCRIPTION OF THE DRAWINGS

FIG. 1 depicts a programmed bioelectric stimulator for delivery to a subject connected to multiple soft conductive electrode pads.

FIG. 2 depicts a programmed bioelectric stimulator as described herein.

FIG. 3 depicts a conductive soft wrap for use with the system.

FIG. 4 depicts a programmed bioelectric stimulator depicted alongside a pen.

FIG. 5 depicts a bioelectric stimulation system.

### DETAILED DESCRIPTION

Inoculants for use with the methods described herein include various vaccines being developed to prevent COVID-19. For instance, the vaccine currently called “ChAdOx1 nCoV-19,” popularly known as the “Oxford vaccine,” is being developed by Oxford University in collaboration with pharmaceutical company AstraZeneca. The vaccine is made from a weakened version of adenovirus, which infects chimpanzees. It has been genetically altered so that it does not replicate in humans and has added genes to code for the so-called spike proteins that the coronavirus uses to infect human cells.

Similarly, CanSino Biologics, in collaboration with the Beijing Institute of Biotechnology, developed a vaccine using a weakened adenovirus. Unlike the Oxford vaccine, which relies on an adenovirus that infects chimpanzees, CanSino Biologics, inter alia, is using an adenovirus that infects humans.

Likewise, Johnson & Johnson's Janssen experimental COVID-19 vaccine was also developed from a weakened adenovirus (Ad26). This type of vaccine is called a vector-based vaccine because it uses a weakened virus (a vector) to deliver information about the pathogen to the body to spur the immune response. In this case, the weakened adenovirus expresses the SARS-CoV-2 “spike” protein. Mercado et al., “Single-shot Ad26 vaccine protects against SARS-CoV-2 in rhesus macaques,” Nature (Jul. 30, 2020); doi.org/10.1038/s41586-020-2607-z.

Another vaccine, called “PiCoVacc” was developed by Beijing-based Sinovac Biotech, protected rhesus macaque monkeys from infection with the novel coronavirus. Gao et al., “Development of an inactivated vaccine candidate for SARS-CoV-2,” Science, Vol. 369, Issue 6499, pp. 77-81 (July 2020), the contents of which are incorporated herein by this reference.

Similarly, China National Pharmaceutical Group's (“Sinopharm's”) candidate vaccine is also an inactivated form of SARS-CoV-2.

Another vaccine (“mRNA-1273”), was developed by U.S. biotech company Moderna and the National Institute of Allergy and Infectious Diseases (NIAID). Pfizer and German biotechnology company BioNTech are, like Moderna, developing a vaccine that uses messenger RNA to prompt the immune system to recognize the coronavirus.

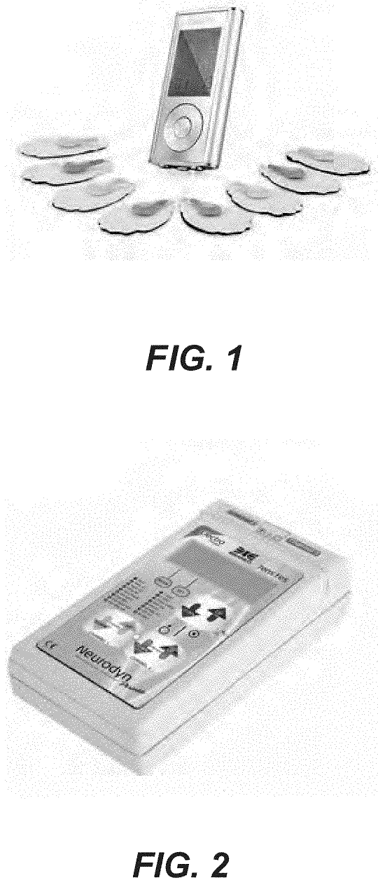

A bioelectric stimulator (see, e.g., FIGS. 1, 2, and 4) that upregulates expression of stem cell homing factor (“SDF-1”) is disclosed in U.S. Pat. No. 10,695,563 B2 to Leonhardt et al. (Jun. 30, 2020) for “Orthodontic treatment,” the contents of which are incorporated herein by this reference.

SDF-1 recruits natural killer cells, T cells, and neutrophils to an area. Isaacson et al., “Stromal Cell-Derived Factor 1 Mediates Immune Cell Attraction upon Urinary Tract Infection,” Cell Reports vol. 20, pp. 40-47 (2017). SDF-1 may thus be useful in treating a COVID-19 patient.

In certain embodiments, the trainable cells of the immune system are first trained with respect to coronavirus. See, e.g., Kipnis et al., infra, and Kar and Joosten, “Training the trainable cells of the immune system and beyond,” Nature Immunology volume 21, pages 115-119 (2020) (describing “training immunity”), the contents of each of which are incorporated herein by this reference.

In certain embodiments, the described treatment and system is combined with training a subject's T cell and T Helper Cells by vaccine-type exposure to up to three common cold coronaviruses (e.g., common cold coronaviruses HCoV-OC43, HCoV-229E, HCoV-NL63, or HCoV-HKU1) and inducing an immune response against them. A. Woodward, “Common Colds May Have ‘Primed’ Some People's Immune Systems For COVID-19,” Business Insider (Aug. 7, 2020); Mateus et al., “Selective and cross-reactive SARS-CoV-2 T cell epitopes in unexposed humans,” Science 4 Aug. 2020: eabd3871; DOI: 10.1126/science.abd3871.

A T-helper cell is type of T cell that helps other cells in the immune response by recognizing foreign antigens and secreting substances called “cytokines,” which activate T and B cells. T-helper cells generally fall into two main classes: those that activate other T cells for cellular inflammatory responses; and those that drive B cells to produce antibodies in the humoral immune response. These two classes of response are generally incompatible with one another and require coordination by substances called cytokines to promote one response while dampening the other. T-helper cells have CD4 markers on their surface. They are a special subpopulation of CD4 cells.

Sekine et al., supra, reported that “the absolute numbers and relative frequencies of CD4+ and CD8+ T cells were unphysiologically low in patients with acute moderate or severe COVID-19.”

IL-2 acts primarily as a T cell growth factor, essential for the proliferation and survival of T cells as well as the generation of effector and memory T cells. IL-2 is a four α-helical bundle cytokine that belongs to a family of structurally related cytokines that includes IL-4, IL-7, IL-9, IL-15, and IL-21. It acts primarily as a T cell growth factor, essential for the proliferation and survival of T cells as well as the generation of effector and memory T cells.

As described herein, upregulation of expression of IL-2, otherwise known as “T cell growth factor,” stimulate an increase T cells and T helper cells, which identify and attack a virus (e.g., SARS-COV-2) as it enters the subject's body and before it has a chance to take hold. IL-2 upregulation is combined with, for example, a mild vaccine or mild exposure to SARS-COV-2, to trigger a memory T cell response specific to SARS-COV-2. So in simple terms, the patient needs a combination of SARS-CoV-2-specific memory T cells, a large, strong population of T cells and T helper cells, and needs to avoid T cell exhaustion if there is a chance of exposure to a large dose of the virus in order to handle a big long fight with the virus.

Ross & Cantrell, “Signaling and Function of Interleukin-2 in T Lymphocytes,” Annu. Rev. Immunol. 2018 Apr. 26; 36: 411-433; doi: 10.1146/annurev-immunol-042617-053352 described that “the discovery of IL-2 changed the molecular understanding of how the immune system is controlled. IL-2 is a pleiotropic cytokine, and dissecting the signaling pathways that allow IL-2 to control the differentiation and homeostasis of both pro- and anti-inflammatory T cells is fundamental to determining the molecular details of immune regulation. The IL-2 receptor couples to JAK tyrosine kinases and activates the STAT5 transcription factors. However, IL-2 does much more than control transcriptional programs; it is a key regulator of T cell metabolic programs. The development of global phosphoproteomic approaches has expanded the understanding of IL-2 signaling further, revealing the diversity of phosphoproteins that may be influenced by IL-2 in T cells. However, it is increasingly clear that within each T cell subset, IL-2 will signal within a framework of other signal transduction networks that together will shape the transcriptional and metabolic programs that determine T cell fate.”

Cossarizza et al., “Extremely low frequency pulsed electromagnetic fields increase interleukin-2 (ICosL-2) utilization and IL-2 receptor expression in mitogen-stimulated human lymphocytes from old subjects,” FEBS LETTERS, 248(1.2):141-144 (1989), the contents of which are incorporated herein by this reference, describes the effects of exposing mitogen-stimulated human lymphocytes from aged subjects to low-frequency pulsed electromagnetic fields (“PEMFs”), which were studied by measuring the production of interleukin-2 (IL-2) and the expression of IL-2 receptor. PEMF-exposed cultures that presented increased ³H-thymidine incorporation showed lower amounts of IL-2 in their supernatants, but higher percentages of IL-2 receptor-positive cells and of T-activated lymphocytes. Taken together, the data suggested that PEMFs were able to modulate mitogen-induced lymphocyte proliferation by provoking an increase in utilization of IL-2, most likely acting on the expression of its receptor on the plasma membrane.

In certain embodiments, T cells (CD8) are primed with bioelectric expression of IL-12, which in turn creates a higher expression of the IL-2 receptor

In certain embodiments, the ways in which T cells in the immune system recognize and fight viruses in the body are bioelectrically mimicked by stimulating the innate immune system by activating Toll-like receptors (TLR).

In certain embodiments, in vitro priming of tumor-specific or virus-specific CD8 T cells with bioelectric stimulation of IL-12 and IL-2 are utilized to induce a stronger immune response than when in a patient's body, while simultaneously promoting memory T cells quantity and ability to vaccinate the patient via those cells ability to remember the tumor or virus type and to attack it quickly in early stages to prevent the dangerous spread of similar new cancers or infections.

In certain embodiments, bioelectric expression of IL-12 is utilized to enhance the immune system by enhancing sensitivity of IL-2 signaling inside the T-cell and thus lower the need for higher doses of IL-2 in fighting cancers and viruses.

In certain embodiments, CD8 T cells are programmed in culture with bioelectric overexpression of IL-12 and IL-2, and then these cells are transferred into patients with cancer tumors or viruses to illicit a strong immune response.

In certain embodiments, brain cancer is targeted through the blood brain barrier by IL-12 and IL-2 receptor bioelectric modification and CD8 T cells and T memory cells are trained to elicit a strong targeted immune response.

The treatment(s) herein described may be further combined with other treatments such as nutritional supplementation. See, e.g., Iddir et al., “Strengthening the Immune System and Reducing Inflammation and Oxidative Stress through Diet and Nutrition: Considerations during the COVID-19 Crisis,” Nutrients. 2020 June; 12(6): 1562; doi: 10.3390/nu12061562.

The therapy may also be combined with anti-coagulant therapy or with testing patients for endothelial cell injury. See, e.g., Goshua et al., “Endotheliopathy in COVID-19-associated coagulopathy: Evidence from a single-centre, cross-sectional study,” The Lancet Haematology (2020); doi: 10.1016/s2352-3026(20)30216-7, the contents of which are incorporated herein by this reference.

The treatment described herein may also be combined with stem cell therapy. See, e.g., Chen et al., “Pulmonary alveolar regeneration in adult COVID-19 patients,” Cell Res. 30, 708-710 (2020), the contents of which are incorporated herein by this reference. Such therapy is supplemented with the application of appropriate bioelectric signals. See, e.g., U.S. Pat. No. 10,960,206 to Leonhardt et al. (Mar. 30, 2021) for “Bioelectric Stimulator,” the contents of which are incorporated herein by this reference. A bench top stimulator (e.g., a Mettler Model 240 Stimulator from Mettler Electronics of Anaheim, Calif., US) may be programmed with the described bioelectric signals.

Preferably, the treatment includes anti-inflammatory therapy, such as bioelectric therapy conducted with the application of bioelectric signals to counter the risk of a cytokine storm often associated with COVID infection. See, e.g., U.S. Pat. No. 11,110,274 to Leonhardt (Sept. 7, 2021) for “System and method for treating inflammation,” the contents of which are incorporated herein by this reference.

In certain embodiments, a bioelectric stimulator is used to reduce the effects of a cytokine storm. Preferably, such a bioelectric stimulator is programmed to produce bioelectric signals that stimulate target tissue in a subject, wherein the bioelectric signals comprise: (a) a biphasic continuous current of 10 μA with a frequency of 50 Hz; (b) a square, biphasic waveform at 50% duty, wherein the frequency is at least 75 Hz and the signal amplitude is 1.0 V; (c) within 15%, 3 mV with a frequency of about 22 Hz, and a current of about 1 mA, followed by 3 mA; (d) within 15%, a biphasic pulse at 20 Hz, 0.1 V, and a 7.8 ms pulse duration; and (e) 3 mV at 2/100 Hz, alternating frequency, with current of 3 mA, followed by 15 Hz, 1 Gauss EM field, consisting of 5-millisecond bursts with 5-microsecond pulses followed by 200 μs pulse duration at 30 Hz and with current amplitude of 140 mA. A method of using this bioelectric stimulator to treat a subject wherein the subject is undergoing or is at risk of undergoing a cytokine storm comprises administering the bioelectric signals to the subject so as to increase the production of (a) interleukin-6 (IL-6), (b) transforming growth factor beta 1 (TGF-β1), (c) insulin-like growth factor 1 (IGF-1), (d) klotho, and/or (e) tissue necrosis factor (TNF).

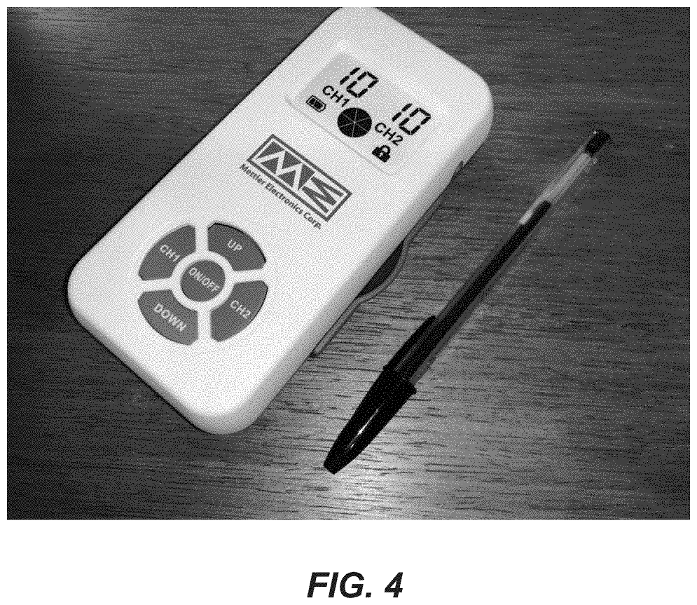

Referring now to FIG. 1, depicted is a stimulator for use in treating a human. The depicted device is about the size of a pen (FIG. 4) and is programmable. A bench top stimulator (e.g., a Mettler Model 240 Stimulator from Mettler Electronics of Anaheim, Calif., US) may be pre-programmed with the bioelectric signaling sequence(s).

The described treatment may further be combined with the tunable control of antibody mobilization. See, e.g., Emaminejad, Sam et al., “Tunable control of antibody immobilization using electric field,” PNAS (USA), vol. 112, 7 (2015): 1995-9. doi:10.1073/pnas.1424592112, the contents of which are incorporated herein by this reference.

The incorporated U.S. Pat. No. 11,110,274 describes a device that measures inflammatory markers in the subject and then the device may be used to deliver at least one bioelectric signal to tissue of the subject so as to, for example, up-regulate expression of selected protein(s), which protein(s) act(s) to balance inflammation in the subject. For example, the device may be used to precisely control (e.g., upregulate) expression of protein, wherein the protein is selected from the group consisting of insulin-like growth factor 1 (“IGF1”), interleukin 6 (“IL-6”), interleukin 10 (“IL-10”), interleukin-1β (“IL-1β”), transforming growth factor-β (“TGFβ”), tumor necrosis factor alpha (“TNF-α”), CXCLS, and any combination thereof.

Among bioelectric signals for other proteins, the incorporated U.S. Pat. No. 11,110,274 also describes particular bioelectric signals for upregulating Activin B (6.0 mV, pulse width 100 μs, square wave), epidermal growth factor (“EGF”) (10 V/cm (5 V here), 500 Hz, pulse width 180 μs, square wave), follistatin (10 V/cm, 50 Hz, square wave), hepatocyte growth factor (“HGF”) (3.5 V, 10 second burst every 30 seconds, square wave), insulin-like growth factor 1 (“IGF1”) (3.0 mV, 22 Hz, square wave), osteoprotegerin (OPG) (4.0 mV, 2,000 Hz, square wave), platelet-derived growth factor (“PDGF”) (30%: 3 V/cm (100 mV depicted), 10 Hz, pulse width 200 μs, square wave), PDGF (230%: 20 V/cm (7.0 V depicted), 100 Hz, pulse width 100 μs, square wave), stem cell proliferation (15 mV, 70 Hz, square wave), stem cell proliferation: (2.5-6.0 V (4 V depicted in U.S. Pat. No. 11,110,274 A1), 20 Hz, pulse width 200-700 μs, square wave), receptor activator of nuclear factor kappa-B ligand (“RANKL”) (3.0 mV, 2 Hz, square wave), Stromal Cell-Derived Factor 1 (“SDF-1”), (3.5 mV, 30 Hz, square wave), tropoelastin (60 mV, 50 Hz, square wave), vascular endothelial growth factor (“VEGF”) (100 mV, 50 Hz, square wave), and SDF-1 (2nd part) (0.25 mA (3.0 V depicted in U.S. Pat. No. 11,110,274 A1), 100 Hz, 100 μs pulse width, square wave).

A biphasic continuous current of 10 μA with a frequency of 50 Hz upregulates the expression of IL-6. Compare Spadari et al., “Electrical stimulation enhances tissues reorganization during orthodontic tooth movement in rats,” Clin. Oral Investig. 2017; 21:111-120. DOI: 10.1007/s00784-016-1759-6, the contents of which are incorporated herein by this reference.

Extremely low frequency pulsed electromagnetic fields increase interleukin-2 (IL-2) utilization and IL-2 receptor expression in mitogen-stimulated human lymphocytes from 86 to 90 year old subjects. Cossarizza et al. supra.

Bioelectric signals for upregulating expression of klotho are described in U.S. Patent Application Publication US 2020-0289826-A1 to Leonhardt et al. (Sep. 17, 2020) for “Klotho Modulation,” the contents of which are incorporated herein by this reference. Klotho is known to improve mucociliary clearance in the lung. See, e.g., Garth et al., “The Effects of the Ant-aging Protein Klotho on Mucociliary Clearance,” Front. Med. (Jan. 24, 2020).

The application of harmonic vibrational energy delivered into the patient's lungs to prevent blood clot aggregation is also contemplated. See, e.g., U.S. Pat. No. 5,788,668 to Leonhardt et al. (Aug. 4, 1998) for “Vibrational enhancement of intravenous gas exchanging devices and long-term intravenous devices,” the contents of which are incorporated herein by this reference. There is described a method where a programmable signal source produces a desired output signal that is transferred by a conduit means or conducting means into a patient by percutaneous venous insertion. The output signal is either vibrational or electrical. If vibrational, the conduit means or one or more transducers radiates the output signal into the treatment site within a patient. If electrical, one or more transducers receive the output signal and convert the output signal into vibration and then radiate it into the treatment site within a patient. The treatment site is the location of a catheter or other intravenous device, residing within the patient for the purposes of gas exchange in the blood stream or for other long-term treatment. The presence of the vibration increases the efficiency of intravenous gas exchanging devices significantly, and prevents clot formation on the surface of intravenous devices.

Also contemplated is the use of Raman spectroscopy RNA light change detection to assist in the custom design of bioelectric signaling sequences for treatment of COVID-19. Weintraub et al. (2020) infra. Such a use may be based upon surface enhanced Raman spectroscopy (“SERS”). Developed by Dr. Laura Fabris, SERS “is a sensitive method that detects interactions between molecules through changes in how they scatter light. [R]esearchers decided to use the method to study influenza A. To detect the virus's RNA, they added to gold nanoparticles a ‘beacon DNA’ specific to influenza A. In the presence of influenza A RNA, the beacon produced a strong SERS signal, whereas in the absence of this RNA, it did not. The beacon produced weaker SERS signals with increasing numbers of viral mutations, allowing the researchers to detect as few as two nucleotide changes. Importantly, the nanoparticles could enter human cells in a dish, and they produced a SERS signal only in those cells expressing influenza A RNA.” See, e.g., “Studying viral outbreaks in single cells could reveal new ways to defeat them (video)” (Aug. 20, 2020); www.acs.org/content/acs/en/pressroom/newsreleases/2020/august/studying-viral-outbreaks-in-single-cells-could-reveal-new-ways-to-defeat-them-video.html, the contents of which are incorporated herein by this reference. Such a use includes a method of selecting bioelectric signaling sequences to treat a subject suffering from COVID-19, the method comprising utilizing Raman spectroscopy RNA light change detection to assist in the selection of bioelectric signals to treat the subject.

In certain embodiments, mild electrical stimulation is utilized to reduce the severity of a “cytokine storm” the COVID patient may be suffering. For example, mild electrical stimulation with high frequency pulse-current (5500 pulse per second) has been shown to suppress the overproduction of pro-inflammatory cytokines. See, e.g., Piruzyan et al., “A novel condition of mild electrical stimulation exerts immunosuppression via hydrogen peroxide production that controls multiple signaling pathway,” PLoS ONE 15(6): e0234867 (2020). doi.org/10.1371/journal.pone.0234867, the contents of which are incorporated herein by this reference.

The invention is further described with the aid of the following illustrative EXAMPLES.

### EXAMPLE I

A randomized controlled trial is conducted having approximately 20 subjects. The duration of the study is 2 to 3 weeks. Eligibility Criteria include: age 18-80 years old; diagnosis of COVID-19, in mechanical ventilation, acute respiratory distress syndrome, and Using muscle blocker at the first moment. Exclusion Criteria include: patients who have an important sensitivity alteration; epidermal lesions at the application site; patients with pulmonary thromboembolism and thrombophlebitis; patients with pacemakers; patients with cardiac arrhythmia; patients with hemodynamic instability (MAP <60 mmHg); patients with femoral venous access (Permcath catheter); patients with an intra-aortic balloon; obese patients (BMI≥30); patients on continuous dialysis; feverish state; patients with epilepsy; and pregnancy.

A Mettler Model 240 or similar FDA-approved stimulator is used with the following Protocols: Protocol 1: 30 minutes (sensory stimulation—kidneys); Protocol 2: 15 to 30 minutes (motor stimulation—quadriceps muscle).

Stimulation is once a day; from admission to discharge from the ICU (according to the number of days the patient remains in the ICU (on average 15-21 days)).

First, the patients allocated to the intervention group will receive Protocol 1 and after removal of the muscle blocker they will receive the protocol 1+2. The control group will not receive any intervention, only ICU routine physiotherapy.

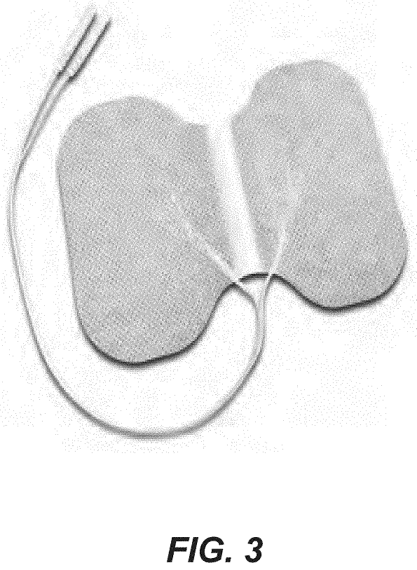

For direct stimulation of the kidneys and alpha klotho protein, the electrodes (see, e.g., FIGS. 1 and 3) will be placed in the abdominal area corresponding to the kidney anatomical site and dorsal region at the level of the 10th thoracic vertebra.

The parameters used will be: Current: symmetrical biphasic pulsed (TENS); Frequency: 20 Hz; Pulse width: 1000 microseconds; and Intensity: it will be increase progressively (every session) until reaching the limit of the sensory threshold.

Primary Outcomes: Kidney function and systemic inflammation by a-klotho protein expression, creatinine, IL-2, IL-6, IL-10, TNFα and C-reactive protein. [Time Frame: Baseline and weekly, until discharge from the ICU or death].

Secondary Outcomes: Muscle damage assessed by creatine kinase (CK) dosage. [Time Frame: Baseline and weekly, until discharge from the ICU or death]; Functionality assessed using the scale Perme Intensive Care Unit Mobility Score. [Time Frame: Baseline and weekly, until discharge from the ICU or death]; Lower limb muscle strength through scale Medical Research Council (MRC). [Time Frame: After withdrawal of sedation weekly until discharge from the ICU or death].

### EXAMPLE II

70 subjects diagnosed as having Covid-19 are studied. One group is administered stimulation with a bioelectric signal having a biphasic pulse of 20 Hz with a 7.8 millisecond pulse duration (0.1 V as measured at the level of the cells being stimulated), which upregulates expression of klotho, in comparison to a control group. The patients are stimulated over their respective kidneys and on the thighs.

Results: In comparison to the control patents, there is a 20% reduction in ventilator time and a 20 to 30% reduction of ICU time for the group receiving the bioelectric stimulation.

### EXAMPLE III

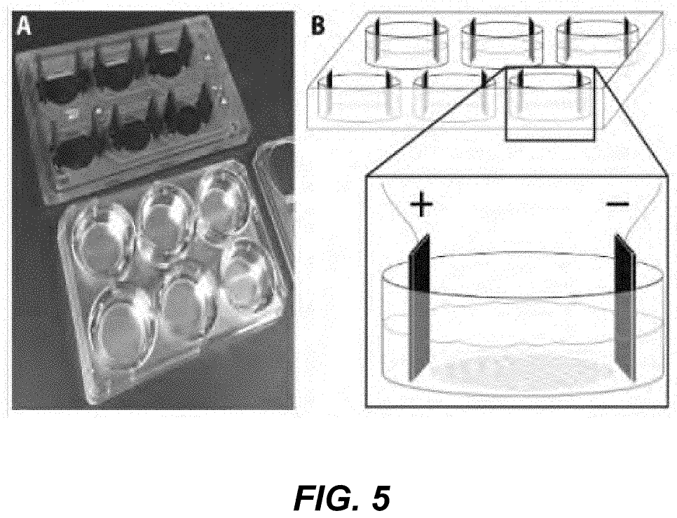

A bioelectric stimulator producing a bioelectric signal of 20 V, biphasic current of frequency 1 Hz and pulse width duration of 5 ms was applied (see, e.g., FIG. 5) to bone marrow-derived mesenchymal stem/stromal cells (BMSC) and adipose tissue-derived stem cells (ADSC) for 24 hours, and expression levels of interferon type 1 (IFN-1), interferon β (IFNβ), sphingosine kinase 1 (SPHK1), AKT-1, Angiopoietin 2 (ANGPT-2), B-cell lymphoma 2 (BCL-2), chemokine (C-X-C motif) ligand 9 (CXCL9), chemokine (C-X-C motif) ligand 10 (CXCL10), basic fibroblast growth factor (FGF-β or FGF-2), leptin (LEP), transforming growth factor-beta 2 (TGF-β2), and transforming growth factor (TGF-β1) receptor were measured.

FIG. 5 depicts a bioelectric stimulation system in which cells and/or tissue may be plated in each dish and cultured. Stimulation occurs using an electrode array (shown at the top of panel A), which is inverted and introduced into the 6-well dish where cells are grown. Each well receives uniform stimulation via a pair of carbon electrodes.

The expression of the following were upregulated: IFN-1 (740% in BMSC and 1,120% in ADSC after 24 hours of bioelectric signal stimulation), IFNβ (1,560% in BMSC and 2822% in ADSC after 12 to 24 hours of bioelectric signal stimulation), and SPHK1 (410% in BMSC and 240% in ADSC after 12 to 24 hours of bioelectric signal stimulation).

The expression of the following were downregulated: AKT-1 (245% in ADSC after 15 to 30 minutes of bioelectric signal stimulation), ANGPT-2 (290%-300% in ADSC after 15 minutes (300%) to 3 hours (290%) of bioelectric signal stimulation), BCL-2 (200%-230% in ADSC after 3 hours and 24 hours of bioelectric signal stimulation), CXCL9 (290%-300% in ADSC after 15 minutes to 1 hour of bioelectric signal stimulation), CXCL10 (300%-468% in ADSC after 15 minutes (200 to 360%), 30 minutes (468%), and 1 hour (420%) of bioelectric signal stimulation), FGF-β (432% in ADSC after 12 to 24 hours of bioelectric signal stimulation), LEP (226% in BMSC and 387% in ADSC after 3 hours of bioelectric signal stimulation), TGF-β2 (230% in BMSC and 530% in ADSC after 12 to 24 hours of bioelectric signal stimulation), and TGF-β1 (203% in ADSC after 12 to 24 hours of bioelectric signal stimulation).

### EXAMPLE IV

The bioelectric stimulator of EXAMPLE III is further programmed to produce a bioelectric signal of, within 15%, a biphasic current of frequency 20 Hz and a 7.8 ms pulse duration and/or produce at least one bioelectric signal having a frequency selected from the group consisting of 5 Hz, 10 Hz, 20 Hz, 25 Hz, 50 Hz, 75 Hz, 100 Hz, 250 Hz, 500 Hz, 750 Hz, 2,500 Hz, 100,000 Hz, 500,000 Hz, and 1 MHz. U.S. Patent Application Publication US 2020-0289826-A1 to Leonhardt et al. (Sep. 17, 2020) for “Klotho Modulation,” and U.S. patent application Ser. No. 17/473,809 to Leonhardt, filed Sep. 13, 2021. In addition to the modulation of proteins described in EXAMPLE III, expression of klotho is upregulated in COVID patients who are treated with the bioelectric stimulator of this EXAMPLE IV.

### REFERENCES

(The contents of each of which are incorporated herein by this reference.)
Bachmann et al., “Interleukin 2: from immunostimulation to immunoregulation and back again,” EMBO Rep. 2007 December; 8(12): 1142-1148; doi: 10.1038/sj.embor.7401099.
Chen W., “A potential treatment of COVID-19 with TGF-β blockade,” Int. J. Biol. Sci. 2020 Apr. 21; 16(11):1954-1955. Doi: 10.7150/ijbs.46891. PMID: 32398962; PMCID: PMC7211163.
Cossarizza et al., “Extremely low frequency pulsed electromagnetic fields increase interleukin-2 (IL-2) utilization and IL-2 receptor expression in mitogen-stimulated human lymphocytes from old subjects,” FEBS LETTERS, 248(1.2):141-144 (1989).
Emaminejad, Sam et al., “Tunable control of antibody immobilization using electric field,” Proceedings of the National Academy of Sciences of the United States of America vol. 112, 7 (2015): 1995-9. doi:10.1073/pnas.1424592112.
Garth et al., “The Effects of the Ant-aging Protein Klotho on Mucociliary Clearance,” Front. Med. (Jan. 24, 2020).
Ghazavi et al., “Cytokine profile and disease severity in patients with COVID-19,” Cytokine, 2020 Sep. 30; 137:155323. Doi: 10.1016/j.cyto.2020.155323. Epub ahead of print. PMID: 33045526; PMCID: PMC7524708.
Goshua et al., “Endotheliopathy in COVID-19-associated coagulopathy: Evidence from a single-centre, cross-sectional study,” The Lancet Haematology (2020); doi:10.1016/s2352-3026(20)30216-7.
Hanna et al., “Evaluation of the Role of Hedgehog Interacting Protein (HHIP) and the Sonic Hedgehog Pathway to Enhance Respiratory Repair and Function in Chronic Obstructive Pulmonary Disease (COPD),” American Journal of Respiratory and Critical Care Medicine 2020; 201:A4062.
Hoffmann & Gill, “Externally Applied Vibration at 50 Hz Facilitates Dissolution of Blood Clots In-Vitro,” Am. J. Biomed. Sci. 2012, 4(4), 274-284; doi: 10.5099/aj120400274.
Iddir et al., “Strengthening the Immune System and Reducing Inflammation and Oxidative Stress through Diet and Nutrition: Considerations during the COVID-19 Crisis,” Nutrients. 2020 June; 12(6): 1562; doi: 10.3390/nu12061562.
Isaacson et al., “Stromal Cell-Derived Factor 1 Mediates Immune Cell Attraction upon Urinary Tract Infection,” Cell Reports vol. 20, pp. 40-47 (2017).
Jiang S, Sakamoto R, Kimura T., “A guinea pig IFNA1 gene with antiviral activity against human influenza virus infection,” Front Biosci. (Landmark Ed). 2019 March 1; 24:790-797. PMID: 30844713.
Kar and Joosten, “Training the trainable cells of the immune system and beyond,” Nature Immunology volume 21, pages 115-119 (2020).
Kennedy et al., “Impaired innate, humoral, and cellular immunity despite a take in smallpox vaccine recipients,” Vaccine, 2016 Jun. 14; 34(28):3283-90. Doi: 10.1016/j.vaccine.2016.05.005. Epub 2016 May 11. PMID: 27177944; PMCID: PMC5528000.
Khan et al., “The Timing of Stimulation and IL-2 Signaling Regulate Secondary CD8 T Cell Responses,” PLoS Pathog. 2015 October; 11(10): e1005199.
Kipnis et al., “Pro-cognitive properties of T cells,” Nat. Rev. Immunol. 2012 September; 12(9): 663-669.
Kumar et al., “Human Sertoli cells support high levels of Zika virus replication and persistence,” Sci. Rep. 2018 Apr. 3; 8(1):5477. doi: 10.1038/s41598-018-23899-x. PMID: 29615760; PMCID: PMC5883016.
M. Leslie, “T cells found in COVID-19 patients ‘bode well’ for long-term immunity,” ScienceMag.org (May 14, 2020).
Limonta et al., “Fibroblast Growth Factor 2 Enhances Zika Virus Infection in Human Fetal Brain,” J. Infect. Dis. 2019 Sep. 13; 220(8):1377-1387. doi: 10.1093/infdis/jiz073. PMID: 30799482; PMCID: PMC6743838.
C. Lytal, “USC Stem Cell scientists use ‘mini-lungs’ and lung models to understand COVID-19,” USC Stem Cell (Jun. 27, 2020); stemcell.keck.usc.edu/usc-stem-cell-scientists-use-mini-lungs-and-lung-models-to-understand-covid-19/.
Madhuri et al., “BCG vaccine and COVID-19: implications for infection prophylaxis and cancer immunotherapy,” J. Immunother. Cancer. 2020 July; 8(2):e001119. Doi: 10.1136/jitc-2020-001119. PMID: 32636240; PMCID: PMC7342862.
Mateus et al., “Selective and cross-reactive SARS-CoV-2 T cell epitopes in unexposed humans,” Science 4 Aug. 2020: eabd3871; DOI: 10.1126/science.abd3871.
McGowan et al., “Targeting the SphK-S1P-SIPR Pathway as a Potential Therapeutic Approach for COVID-19,” Int. J. Mol. Sci. 2020 Sep. 29; 21(19):7189. Doi: 10.3390/ijms21197189. PMID: 33003377; PMCID: PMC7583882.
Piruzyan et al., “A novel condition of mild electrical stimulation exerts immunosuppression via hydrogen peroxide production that controls multiple signaling pathway,” PLoS ONE 15(6): e0234867 (2020). doi.org/10.1371/journal.pone.0234867.
R. Rettner, “Common colds train the immune system to recognize COVID-19,” www.livescience.com/common-cold-coronaviruses-t-cells-covid-19-immunity.html (August 2020).
Ross & Cantrell, “Signaling and Function of Interleukin-2 in T Lymphocytes,” Annu. Rev. Immunol. 2018 Apr. 26; 36: 411-433; doi: 10.1146/annurev-immunol-042617-053352.
“Scientists discover way to amp up power of killer T cells to fight melanoma,” Science Daily (May 11, 2011). www.sciencedaily.com/releases/2011/05/110510101722.htm.
Sekine et al., “Robust T cell immunity in convalescent individuals with asymptomatic or mild COVID-19,” bioRxiv (Jun. 29, 2020).
Simone et al., “Intussusceptive angiogenesis in Covid-19: hypothesis on the significance and focus on the possible role of FGF2,” Mol. Biol. Rep. 2020 October; 47(10):8301-8304. Doi: 10.1007/s11033-020-05831-7. Epub 2020 Sep. 12. PMID: 32920756; PMCID: PMC7486971.
Spadari et al., “Electrical stimulation enhances tissues reorganization during orthodontic tooth movement in rats,” Clin. Oral Investig. 2017; 21:111-120. DOI: 10.1007/s00784-016-1759-6.
Sbruzzi and Plentz, “Indication and Use of Neuromuscular Electrical Stimulation (Nmes) in the Treatment of Critical Adult Patients with COVID-19,” ASSOBRAFIR Ciência, vol. 11, Suplemento 1, p. 133-142, 2020; DOI:dx.doi.org/10.47066/2177-9333.AC20.covid19.013.
A. Weintraub, “Outsmarting COVID-19 and other viruses by analyzing RNA in single cells,” FIERCE Biotech Special Report (Aug. 21, 2020); www.fiercebiotech.com/research/outsmarting-covid-19-and-other-coronaviruses-by-analyzing-rna-single-cells?mkt_tok=eyJpIjoiW1RRMVpETmxPREprTmprMyIsInQiOiJjRUQrbnZEMkx 3UWRCM2xvbzMxSVFQQ1lPa1ZndTZ2VDVDU3I0c09XTkdOYzJ1dTV1NWh4UGw1Nzlue ExhSlwvSkRUbzlcL2VpQWF4UDdPYXJ0dG9wUDRZU1VaY2RkOVRneW5HM0hUMURrb kozc2Y3WjIxQmpDZGtsR1BBa2FSb1F6In0%3D&mrkid=669951.
Zhang et al., “Potential contribution of increased soluble IL-2R to lymphopenia in COVID-19 patients,” Cell Mol. Immunol. 17, 878-880 (2020).
Zhang et al., “Discharge may not be the end of treatment: Pay attention to pulmonary fibrosis caused by severe COVID-19,” J. Med. Virol. 2020 Oct. 27. Doi: 10.1002/jmv.26634. Epub ahead of print. PMID: 33107641.
U.S. Patent Application Publication US 2020-0324106-A1 to Leonhardt et al. (Oct. 15, 2020) for “Bioelectric Stimulation for Sonic Hedgehog Expression.”
U.S. Pat. No. 5,788,668 to Leonhardt (Aug. 4, 1998) for “Vibrational enhancement of intravenous gas exchanging devices and long-term intravenous devices.”
U.S. Pat. No. 10,695,563 B2 to Leonhardt et al. (Jun. 30, 2020) for “Orthodontic treatment.”
U.S. Pat. No. 10,960,206 to Leonhardt et al. (Mar. 30, 2021) for “Bioelectric Stimulator.”
U.S. Pat. No. 11,110,274 to Leonhardt (Sep. 7, 2021) for “System and method for treating inflammation.”
U.S. Patent Application Publication US 2020-0289826-A1 to Leonhardt et al. (Sep. 17, 2020) for “Klotho Modulation” and U.S. patent application Ser. No. 17/473,809 to Leonhardt, filed Sep. 13, 2021.
EP 3,262,159 B1 (Jul. 24, 2019) to Gunther et al., for “Genetically Modified Mesenchymal Stem Cell Expressing Klotho.”

1. A method of treating a mammalian subject who is intending to undergo exposure to an inoculant comprising a virus, polynucleotide(s) encoding at least a portion of the virus, and/or epitope(s) of the virus, the method comprising:
administering at least one bioelectric signal to the subject before, during, and/or immediately after exposure to the inoculant in such a manner as to increase the subject's T cell count and/or T helper cell count.

2. The method according to claim 1, further comprising:
inoculating the subject with the inoculant after administration of the at least one bioelectric signal and after the subject has experienced an increased T cell count and/or T helper cell count so as to create specific memory T cells against the virus.

3. The method according to claim 1, wherein the virus is SARS-CoV-2.

4. The method according to claim 2, wherein the virus is SARS-CoV-2.

5. The method according to claim 1, further comprising:
training a subject's T cell and T Helper Cells by vaccine-type exposure to up to three common cold coronaviruses, and
inducing an immune response against said common cold coronavirus(es).

6. The method according to claim 5, wherein the common cold coronaviruses are selected from the group consisting of HCoV-OC43, HCoV-229E, HCoV-NL63, and HCoV-HKU1.

7. The method according to claim 1, wherein the bioelectric signal is applied near the subject's thyroid.

8. The method according to claim 1, wherein the bioelectric signal upregulates expression of at least one of stromal cell-derived factor 1 (SDF-1), interleukin-2 (IL-2), interleukin-12 (IL-12), interferon type 1 (INF-1), interferon β (IFNβ), sphingosine kinase 1 (SPHK1), klotho, or any combination thereof.

9. The method according to claim 1, further comprising:
administering to the subject a material that stimulates an immune response to SARS-CoV-2, enhances function of the subject's T cells and/or reduces T cell exhaustion, and/or induces an immune response against spike protein of SARS-CoV-2.

10. The method according to claim 1, wherein the inoculant is a polynucleotide comprising mRNA.

11. A method of treating a mammalian subject undergoing a viral infection, the method comprising:
administering bioelectric signals to the subject so as to upregulate expression of SDF-1 in the subject, upregulate expression of PDGF in the subject, upregulate stem cell proliferation in the subject, and upregulate expression of klotho in the subject;
reducing inflammation in the subject, and
administering bioelectric signals to the subject so as to upregulate tissue regeneration in the subject's lungs and blood vessels.

12. The method according to claim 11, wherein a pharmacological agent is administered to the subject so as to reduce inflammation and/or at least one bioelectric signal is administered to the subject for inflammation reduction in the subject.

13. The method according to claim 11, wherein the virus is SARS-CoV-2.

14. The method according to claim 11, further comprising:
applying harmonic vibrational energy delivered into the subject's lungs.

15. The method according to claim 11, further comprising:
administering to the subject a composition comprising materials selected from the group consisting of hypoxia-treated mesenchymal stem cells (“MSCs”), klotho-expressing MSCs, stromal fraction, lung matrix, exosomes, micro RNA gel, selected alkaloids, nutrient hydrogel, bioelectric treated platelet rich fibrin, amniotic fluid, secretome from amniotic sourcing, Wharton's Jelly, growth factors, proteins, and combinations of any thereof.

16. A bioelectric stimulator programmed to produce bioelectric signals that stimulate target tissue in a subject, wherein the bioelectric signals comprise:
(a) a biphasic continuous current with a frequency of 50 Hz;
(b) a square, biphasic waveform at 50% duty, wherein the frequency is at least 75 Hz;
(c) within 15%, a frequency of about 22 Hz;
(d) within 15%, a biphasic pulse at 20 Hz, and a 7.8 ms pulse duration; and
(e) 2/100 Hz, alternating frequency, followed by 15 Hz, 1 Gauss EM field, consisting of 5-millisecond bursts with 5-microsecond pulses followed by 200 μs pulse duration at 30 Hz.

17.-21. (canceled)

22. A method of modulating expression of at least one protein in a subject's tissue, wherein the protein is selected from the group consisting of interferon type 1 (IFN-1), interferon β (IFNβ), sphingosine kinase 1 (SPHK1), AKT-1, angiopoietin 2 (ANGPT-2), B-cell lymphoma 2 (BCL-2), chemokine (C-X-C motif) ligand 9 (CXCL9), chemokine (C-X-C motif) ligand 10 (CXCL10), basic fibroblast growth factor (FGF-β), leptin (LEP), transforming growth factor-beta 2 (TGF-β2), transforming growth factor (TGF-β1) receptor, and any combination thereof, the method comprising:
using a bioelectric stimulator programmed to produce at least one bioelectric signal of, within 15%, a biphasic current of frequency 1 Hz and pulse width duration of 5 ms to deliver the bioelectric signal(s) to the subject's tissue so as to modulate expression of said selected protein(s) by the tissue.

23. The method according to claim 22, wherein modulating expression of at least one selected protein comprises inhibiting expression of AKT-1, ANGPT-2, BCL-2, CXCL9, CXCL10, FGF-β, LEP, TGF-β2, TGF-β1 receptor, or any combination thereof by the subject's tissue.

24. The method according to claim 22, wherein modulating expression of at least one selected protein comprises upregulating expression of IFN-1, IFNβ, SPHK1, or any combination thereof by the subject's tissue.

25. The method according to claim 22, wherein the subject's tissue comprises muscle tissue and the bioelectric signal is from 2 to 20 mA as may be measured three (3) mm deep into the tissue.

26. The method according to claim 22, wherein the bioelectric stimulator is further programmed to produce a bioelectric signal of, within 15%, a biphasic current of frequency 20 Hz and a 7.8 ms pulse duration and/or at least one bioelectric signal having a frequency selected from the group consisting of 5 Hz, 10 Hz, 20 Hz, 25 Hz, 50 Hz, 75 Hz, 100 Hz, 250 Hz, 500 Hz, 750 Hz, 2,500 Hz, 100,000 Hz, 500,000 Hz, and 1 MHz.
